(19) United States
(12) Patent Application Publication
(10) Pub. No.: US 2003/0212271 A1
(43) Pub. Date: Nov. 13, 2003

(54) Trifluoroacetic acid process to prepare [2,3-B]pyridine intermediates

(76) Inventor: Allen Scott, Kalamazoo, MI (US)

Correspondence Address: PHARMACIA & UPJOHN, 301 HENRIETTA ST, 0228-32-LAW, KALAMAZOO, MI, 49007, US

(21) Appl. No.: 10/409,801

(22) Filed: Apr. 9, 2003

Related U.S. Application Data
(60) Provisional application No. 60/374,545, filed on Apr. 22, 2002.

(51) Int. Cl. C07D 491/02; C07D 498/02; C07D 413/02; C07D 215/36

(52) U.S. Cl. 544/127; 546/114; 546/115; 546/156; 544/128

(57) ABSTRACT

The invention is a process for the preparation of a [2,3-b]pyridine of formula (II)

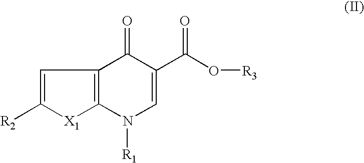

which comprises:

(1) contacting a malonate diester of formula (I)

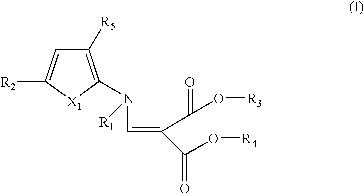

with trifluoroacetic acid; and

(2) adjusting the pH with aqueous base to from about 9 to about 10.5.

### CROSS-REFERENCE TO RELATED APPLICATIONS

This application claims the benefit of the following provisional application: U.S. Serial No. 60/374,545, filed Apr. 22, 2002, under 35 USC 119(e)(i), which is incorporated herein by reference in its entirety.

### BACKGROUND OF THE INVENTION

1. Field of the Invention

The present invention is an improved process to produce [2,3-b]pyridine compound and, in particular, a pharmaceutically useful intermediate known as ethyl 7-methyl-2-(4-morpholinylmethyl)-4-oxo-4,7-dihydrothieno[2,3-b]pyridine-5-carboxylate.

2. Description of the Related Art

International Publication WO 00/53610 based on PCT/US00/05937 discloses a process for the transformation of diethyl 2-{[[3-(tert-butoxycarbonyl)-5-(4-morpholinylmethyl)-2-thienyl](methyl)amino]methylene}malonate (I) to ethyl 7-methyl-2-(4-morpholinylmethyl)-4-oxo-4,7-dihydrothieno [2,3-b]pyridine-5-carboxylate (II) by use of methanesulfonic acid. The process of the present invention does not use methanesulfonic acid and obtains a higher yield in a cleaner reaction.

Malonate Diethyl Esters (I)

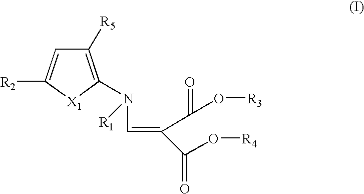

where X₁ is —O—, —S— and —CH═CX₁₋₁— where X₁₋₁ is —H or -F are known, see International Publication WO 00/53610 based on PCT/US00/05937.

More specifically, diethyl 2-{[[3-(tert-butoxycarbonyl)-5-(4-morpholinylmethyl)-2-thienyl](methyl)amino]methylene}-malonate (I) is known, see International Publication WO 00/53610 based on PCT/US00/05937, Preparation 3.

[2,3-b]Pyridines (II)

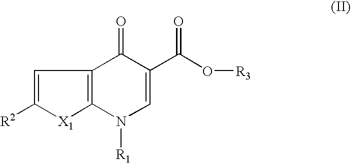

where X₁, R₁, R₂ and R₃ are as defined above are known, see International Publication WO 00/53610. More particularly, ethyl 7-methyl-2-(4-morpholinylmethyl)-4-oxo-4,7-dihydrothieno[2,3-b]pyridine-5-carboxylate (II) is known, see International Publication WO 00/53610, Preparation 4.

The chemical transformation of malonate diethyl esters (I) to [2,3-b]pyridines (II) is known, see International Publication WO 00/53610. More specifically, the chemical transformation of diethyl 2-{[[3-(tert-butoxycarbonyl)-5-(4-morpholinylmethyl)-2-thienyl](methyl)amino]methylene}malonate (I) to ethyl 7-methyl-2-(4-morpholinylmethyl)-4-oxo-4,7-dihydrothieno[2,3-b]pyridine-5-carboxylate (II) is known, see International Publication WO 00/53610, Preparation 4.

The transformation of ethyl 7-methyl-2-(4-morpholinylmethyl)-4-oxo-4,7-dihydrothieno[2,3-b]pyridine-5-carboxylate (II) to N-(4-chlorobenzyl)-7-methyl-2-(4-morpholinylmethyl)-4-oxo-4,7-dihydrothieno[2,3-b]pyridine-5-carboxamide is also known, see International Publication WO 00/53610, Preparations 5 and 6.

### SUMMARY OF INVENTION

Disclosed is a process to prepare a [2,3-b]pyridinyl compound of formula (II)

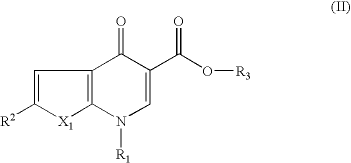

where X₁ is:

—O—,

—S— and

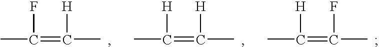

where R₁ is:

C₁-C₄ alkyl,

phenyl and

2-, 3- or 4-pyridinyl;

where R₂ is:

H—,

morpholin-4-yl-CH₂— and

(CH₃)₂N—CH₂—;

where R₃ is C₁-C₄ alkyl; which comprises:

(1) contacting a malonate diester of formula (I)

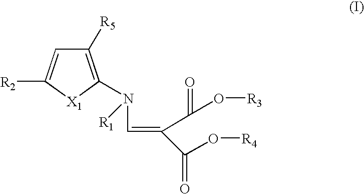

where R₄ is C₁-C₄ alkyl;

where R₅ is:

H—,

HOOC—,

R₅₋₁O—OC— where R₅₋₁ is C₁-C₄ alkyl and

where X₁, R₁, R₂ and R₃ are as defined above, with trifluoroacetic acid; and

(2) adjusting the pH with aqueous base to from about 9 to about 10.5.

### DETAILED DESCRIPTION OF THE INVENTION

In the preferred compounds of formula I X₁ is —S—. It is preferred that R₁ is C₁ alkyl. It is also preferred that R₂ is R₂ is 4-morpholinylmethyl, and that R₃ is C₂ alkyl. It is preferred that R₄ is C₂ alkyl. It is preferred that R₃ and R₄ are the same. It is preferred that R₅ is (CH₃)₃C—O—CO—.

In the process of the present invention the malonate diethyl ester (I) is contacted with trifluoroacetic acid (TFA). The process is practiced by first preparing a mixture of phosphorous pentoxide (P₂O₅) in TFA. Separately, the malonate diethyl ester (I) is dissolved in a suitable solvent such as toluene keeping the temperature in the 15-25° range. To achieve high yields the starting material mixture should be added to the TFA mixture rather than the other way around. During the addition, the temperature should not exceed about 25°; preferred is about 15° to about 25°. The temperature of the reaction mixture should not exceed about 38°; a preferred temperature range is from about 33 to about 36°. The reaction is monitored, preferably hourly, by any of the usual methods such as TLC or HPLC. When the reaction is complete, the TFA is removed by distillation using 25-28 inches of mercury vacuum with a jacket temperature of 60°. Following removal of the TFA, water is added and the pH adjusted to about 9 to about 10.5 preferably from about 9 to about 10 by the addition of base such as hydroxide. The pH must be kept at less than about 10.5. The temperature during the water addition and pH adjustment must be less than 40° to minimize impurity formation; preferred temperatures are from about 15 to about 35°. During the entire process to maximize the yield and minimize byproduct formation the reaction should be agitated fast.

Once the reaction has been quenched, it should not be held for extended periods without adjusting the pH to 9.5-10.5. Storage at low pH will lead to impurity formation.

When the process of the invention is practiced as discussed above, the reaction should be complete in about 2 hours.

The [2,3-b]pyridines (II) are known to be useful intermediates in the production of useful pharmaceutical agents used in the treatment of herpesvirus infections, see International Publication WO 00/53610. More particularly, ethyl 7-methyl-2-(4-morpholinylmethyl)-4-oxo-4,7-dihydrothieno[2,3-b]pyridine-5-carboxylate (II) is known to be a useful intermediate in the production of a useful pharmaceutical agent,

N-(4-chlorobenzyl)-7-methyl-2-(4-morpholinylmethyl)-4-oxo-4,7-dihydrothieno[2,3-b]pyridine-5-carboxamide is known to be a useful pharmaceutical agent in the treatment of a herpesvirus infections, see International Publication WO 00/53610 based on PCT/US00/05937.

### Definitions and Conventions

The definitions and explanations below are for the terms as used throughout this entire document including both the specification and the claims.

Diethyl 2-{[[3-(tert-butoxycarbonyl)-5-(4-morpholinylmethyl)-2-thienyl](methyl)amino]methylene}malonate (Ia) is also known as N-(3-tert-butoxycarbonyl-5-morpholinomethylthien-2-yl)-methylaminomethylenemalonic acid diethyl ester.

TFA refers to trifluoroacetic acid.

All temperatures are in degrees Celsius.

TLC refers to thin-layer chromatography.

HPLC refers to high-pressure liquid chromatography.

Chromatography (column and flash chromatography) refers to purification/separation of compounds expressed as (support, eluent). It is understood that the appropriate fractions are pooled and concentrated to give the desired compound(s).

NMR refers to nuclear (proton) magnetic resonance spectroscopy, chemical shifts are reported in ppm (d) downfield from TMS.

CMR refers to C-13 magnetic resonance spectroscopy, chemical shifts are reported in ppm (δ) downfield from TMS.

Pharmaceutically acceptable refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability.

When solvent pairs are used, the ratios of solvents used are volume/volume (v/v).

When the solubility of a solid in a solvent is used the ratio of the solid to the solvent is weight/volume (wt/v).

### EXAMPLES

Without further elaboration, it is believed that one skilled in the art can, using the preceding description, practice the present invention to its fullest extent. The following detailed examples describe how to prepare the various compounds and/or perform the various processes of the invention and are to be construed as merely illustrative, and not limitations of the preceding disclosure in any way whatsoever. Those skilled in the art will promptly recognize appropriate variations from the procedures both as to reactants and as to reaction conditions and techniques.

### Example 1

### Ethyl 7-methyl-2-(4-morpholinylmethyl)-4-oxo-4,7-dihydrothieno[2,3-b]pyridine-5-carboxylate (IIa)

Phosphorus pentoxide (7.23 g) is combined with trifluoroacetic acid (110 ml) and this mixture is added to a mixture of diethyl 2-{[[3-(tert-butoxycarbonyl)-5-(4-morpholinylmethyl)-2-thienyl](methyl)amino]methylene}malonate (Ia. International Publication WO 00/53610 based on PCT/US00/05937, Preparation 3, 16.4 g) in toluene (18 g) maintaining a temperature of less than 25°. The mixture is heated to about 35° and stirred for at least 2 hrs until the reaction is judged to be complete. The mixture is distilled under reduced pressure to remove the trifluoroacetic acid. The mixture is quenched with water and then methylene chloride is added. The pH is adjusted to about 10 with potassium hydroxide and the phases are separated. The aqueous phase is back-washed with methylene chloride, and the organic phases are combined and distilled under reduced pressure. Toluene is added and distillation continues to remove methylene chloride. Finally, the slurry is cooled to about −10° to −200, filtered, and washed with cold toluene. The cake is dried at about 55° to give the title compound 9.9 g (87% yield), 94% pure by HPLC; NMR (300 MHz CDCl₃) 8.24, 7.41, 4.36, 3.83, 3.72, 2.52 and 1.39 δ.

### Example 2

### Ethyl 7-methyl-4-oxo-4,7-dihydrothieno[2,3-b]pyridine-5-carboxylate (II)

Phosphorus pentoxide (2.8 g) is combined with trifluoroacetic acid (32.4 ml) and to this mixture is added to a mixture of diethyl 2-{[[3-(tert-butoxycarbonyl)-2-thienyl](methyl)amino]methylene}malonate (1, 5.0 g) in toluene (5 nil) maintaining a temperature of less than 25°. The mixture is heated to about 35° and stirred for at least 2 hrs until the reaction is judged to be complete. The mixture is distilled under reduced pressure to remove the trifluoroacetic acid. The mixture is quenched with water (27 ml) and then methylene chloride (35 ml) is added. The pH is adjusted to about 10 with potassium hydroxide and the phases are separated. The aqueous phase is back-washed with methylene chloride, and the organic phases are combined and distilled under reduced pressure. Toluene is added (25 ml) and distillation continues to remove methylene chloride. Finally, the slurry is cooled to about −10 to −20, filtered, and washed with cold toluene. The cake is dried at about 490 to give the title compound, 2.3 g (75% yield), NMR (400 MHz CDCl₃) 8.24, 7.57, 7.02, 4.36, 3.85, and 1.386; CMR (100 MHz CDCl₃) 170.98, 165.31, 149.89, 146.04, 132.97, 124.68, 118.64, 114.88, 60.78, 42.95 and 14.31.

### Example 3

Ethyl 1-methyl-6-(4-morpholinylmethyl)-4-oxo-1,4-dihydro-3-quinolinecarboxylate (II) Phosphorus Pentoxide (0.85 g) is combined with trifluoroacetic acid (3.9 ml) and to this mixture is added to a solution of diethyl 2-{[N-methyl-4-(4-morpholinylmethyl)anilino]methylene}malonate (1, 1.56 g) in toluene (1.56 ml) maintaining a temperature of less than 35°. The mixture is heated to about 55° and stirred for at least 18 hrs until the reaction is judged to be complete. The mixture is quenched with water (1.6 ml) after methylene chloride (14 ml) is added. The pH is adjusted to about 10 with sodium hydroxide and the phases are separated. The aqueous phase is back-washed with methylene chloride, and the organic phases are combined and distilled under reduced pressure. The solid residue is dried at about 35° under reduced pressure to give the title compound, 1.19 g (90% yield); NMR (400 MHz CDCl₃) 8.42, 7.74, 7.41, 4.38, 3.88, 3.69, 3.62, 2.40 and 1.40 δ; CMR (100 MHz CDCl₃) 174.28, 165.70, 149.38, 138.90, 135.29, 133.50, 128.59, 127.81, 115.77, 110.88, 66.89, 62.45, 60.72, 53.55, 41.29 and 14.37 δ.

### Example 4

### Ethyl 1-methyl-8-fluoro-4-oxo-1,4-dihydro-3-quinolinecarboxylate (II)

Phosphorus pentoxide (0.89 g) is combined with trifluoroacetic acid (4.3 ml) and to this mixture is added to a solution of diethyl 2-{[N-methyl-2-fluoroanilino]methylene}malonate (1, 1.23 g) in toluene (1.23 ml) maintaining a temperature of less than 35°. The mixture is heated to about 55° and stirred for at least 18 hrs until the reaction is judged to be complete. The mixture is quenched with water (1.2 ml) after methylene chloride (11 ml) is added. The pH is adjusted to about 10 with sodium hydroxide and the phases are separated. The aqueous phase is back-washed with methylene chloride, and the organic phases are combined and distilled under reduced pressure. The solid residue is dried at about 35° under reduced pressure to give the title compound, 0.82 g (80% yield). NMR (400 MHz CDCl₃) 8.31-8.35, 7.32-7.42, 7.41,4.39, 4.09 and 1.40 δ; CMR (100 MHz CDCl₃) 172.81, 165.34, 152.27, 151.53, 131.57, 129.1. 125.32, 123.55, 119.45, 110.81, 60.93, 45.95 and 14.35 δ.

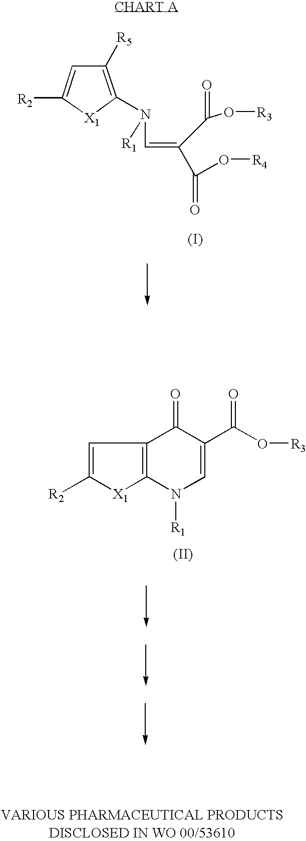

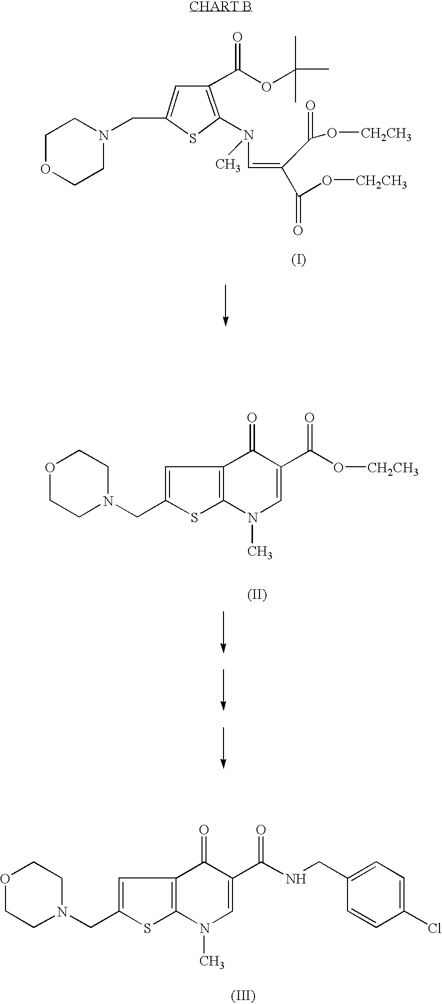

What is claimed is:

1. A process to prepare a [2,3-b]pyridinyl compound of formula (II) 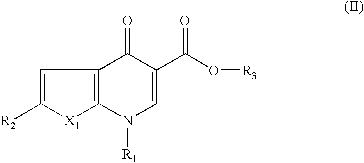
where X₁ is:
—O—,
—S— and 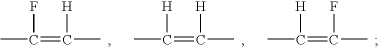
where R₁ is:
C₁-C₄ alkyl,
phenyl and
2-, 3- or 4-pyridinyl;
where R₂ is:
H—,
morpholin-4-yl-CH₂— and
(CH₃)₂N—CH₂—;
where R₃ is C₁-C₄ alkyl; which comprises:
(1) contacting a malonate diester of formula (I) 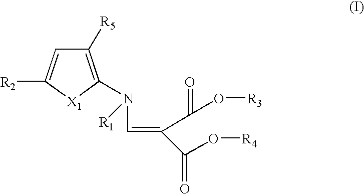
where R₄ is C₁-C₄ alkyl;
where R₅ is:
H—,
HOOC—,
R₅₋₁O—OC— where R₅₋₁ is C₁-C₄ alkyl and
where X₁, R₁, R₂ and R₃ are as defined above, with trifluoroacetic acid; and
(2) adjusting the pH with aqueous base to from about 9 to about 10.5.

2. A process to prepare a [2,3-b]pyridine (II) according to claim 1 where the diethyl malonate ester (1) is added to the trifluoroacetic acid.

3. A process to prepare a [2,3-b]pyridine (II) according to claim 2 where the temperature during the addition of the diethyl malonate ester (I) to the trifluoroacetic acid is less than about 25°.

4. A process to prepare a [2,3-b]pyridine (II) according to claim 3 where the temperature during the addition of the diethyl malonate ester (I) to the trifluoroacetic acid is from about 15° to about 25°.

5. A process to prepare a [2,3-b]pyridine (II) according to claim 1 where the temperature during the contacting of the diethyl malonate ester (I) and the trifluoroacetic acid is less than about 38°.

6. A process to prepare a [2,3-b]pyridine (II) according to claim 5 where the temperature during the contacting of the diethyl malonate ester (I) and the trifluoroacetic acid is from about 33° to about 36°.

7. A process to prepare a [2,3-b]pyridine (II) according to claim 1 where the trifluoroacetic is removed when the reaction is complete and prior to step (2).

8. A process to prepare a [2,3-b]pyridine (II) according to claim 7 where the TFA is removed by distillation.

9. A process to prepare a [2,3-b]pyridine (II) according to claim 1 where water is added when the reaction is complete and prior to step (2).

10. A process to prepare a [2,3-b]pyridine (II) according to claim 1 where the base is aqueous hydroxide.

11. A process to prepare a [2,3-b]pyridine (II) according to claim 1 where the pH is from about 9 to about 10.

12. A process to prepare a [2,3-b]pyridine (II) according to claim 1 where the temperature during the addition of the base is less than about 40°.

13. A process to prepare a [2,3-b]pyridine (II) according to claim 12 where the temperature during the addition of the base is from about 15 to about 35°.

14. A process to prepare a [2,3-b]pyridine (II) according to claim 1 where once the trifluoroacetic acid is removed, the pH is promptly adjusted.

15. A process to prepare a [2,3-b]pyridine (II) according to claim 1 where X₁ is —S—.

16. A process to prepare a [2,3-b]pyridine (II) according to claim 1 where R_{1 is C}₁ alkyl.

17. A process to prepare a [2,3-b]pyridine (II) according to claim 1 where R₂ is 4-morpholinylmethyl.

18. A process to prepare a [2,3-b]pyridine (II) according to claim 1 where R₃ is C₂ alkyl.

19. A process to prepare a [2,3-b]pyridine (II) according to claim 1 where R₄ is C₂ alkyl.

20. A process to prepare a [2,3-b]pyridine (II) according to claim 1 where R₃ and R₄ are the same.

21. A process to prepare a [2,3-b]pyridine (II) according to claim 1 where R₅ is (CH₃)₃C—O—CO—.

22. A process to prepare a [2,3-b]pyridine (II) according to claim 1 where the [2,3-b]pyridine is ethyl 7-methyl-2-(4-morpholinylmethyl)-4-oxo-4,7-dihydrothieno[2,3-b]pyridine-5-carboxylate, ethyl 7-methyl-4-oxo-4,7-dihydrothieno[2,3-b]pyridine-5-carboxylate, ethyl 1-methyl-6-(4-morpholinylmethyl)-4-oxo-1,4-dihydro-3-quinolinecarboxylate and ethyl 1-methyl-8-fluoro-4-oxo-1,4-dihydro-3-quinolinecarboxylate.
